(19) United States
(12) Patent Application Publication
(10) Pub. No.: US 2014/0337040 A1
(43) Pub. Date: Nov. 13, 2014

(54) AUTOMATED SYSTEM FOR MEDICAL ITEM DISPENSING, BILLING, AND INVENTORY MANAGEMENT

(71) Applicants: Brian C. Debusk, Knoxville, TN (US); Angela M. Sewell, Knoxville, TN (US); John G. Jacobs, Knoxville, TN (US); Gregory S. Hodge, Knoxville, TN (US); Kevin E. Lynch, Knoxville, TN (US); William G. Pittman, Knoxville, TN (US)

(72) Inventors: Brian C. Debusk, Knoxville, TN (US); Angela M. Sewell, Knoxville, TN (US); John G. Jacobs, Knoxville, TN (US); Gregory S. Hodge, Knoxville, TN (US); Kevin E. Lynch, Knoxville, TN (US); William G. Pittman, Knoxville, TN (US)

(73) Assignee: DeRoyal Industries, Inc., Powell, TN (US)

(21) Appl. No.: 13/448,732

(22) Filed: Apr. 17, 2012

Related U.S. Application Data
(63) Continuation-in-part of application No. 13/223,641, filed on Sep. 1, 2011, now Pat. No. 8,818,824.

(51) Int. Cl. G06Q 10/08; G06Q 50/22

(52) U.S. Cl. 705/2

(57) ABSTRACT

An inventory management system manages information regarding medical items that are stored in storage containers in a storage structure before being dispensed in conjunction with patient medical treatment. The inventory management system includes inventory sensors associated with the storage containers. Each inventory sensor senses removal or addition of medical items to or from the storage containers, and generates corresponding item inventory signals. Inventory indicia devices are disposed on the storage structure, each associated with a corresponding storage container, and each providing a visual indication that a medical item is to be added or removed. A customer computer, which is in communication with the inventory sensors and inventory indicial devices, runs an inventory application for processing item replenishment information to determine a number of medical items to be added to the storage containers, and activates the inventory indicia devices to visually indicate that medical items are to be added.

This application is a continuation-in-part of and claims priority to co-pending U.S. patent application Ser. No. 13/223,641 filed Sep. 1, 2011, titled AUTOMATED SYSTEM FOR MEDICAL ITEM DISPENSING, BILLING, AND INVENTORY MANAGEMENT.

### FIELD

This invention relates to an inventory management system. More particularly, this invention relates to a system for managing inventories of medical supply items.

### BACKGROUND

Durable Medical Equipment (DME), Prosthetics, Orthotics and Supplies (DMEPOS) as defined by the Department of Health and Human Services and its Center for Medicare Services (CMS) is a class of medical devices, products and supplies that are typically reimbursable under Part B of the U.S. Medicare health care program. In general, this category of products includes items provided to patients who receive outpatient treatment for certain health problems that do not require inpatient admission to a hospital or other healthcare institution. DMEPOS items are typically provided or prescribed to help alleviate, treat or assist in recovery from the condition that prompted the outpatient treatment of the patient. Such outpatient treatment can occur in any number of settings, such as a hospital emergency department, a clinic, or a physician's or therapist's office.

In general, the costs of DMEPOS items are reimbursable or payable separately from the healthcare professional's fee for treatment of the patient. DMEPOS items are typically reimbursable or payable in both Medicare and Medicaid programs and through private health insurers. Traditionally, most DMEPOS items were prescribed by the treating professional and those prescriptions could be filled by DME shops, Orthotics/Prosthetics shops, pharmacies with DME services, etc. However, as a convenience to patients many healthcare providers would like to dispense DMEPOS items at the time of treatment of the patient in order to facilitate patient convenience and continuity of care.

Since DMEPOS items are typically reimbursable or payable under a different billing and reimbursement system than professional healthcare services, it has been difficult for healthcare professionals to provide the dispensing of these items as an adjunct service to their patients. The specialty shops that have traditionally dispensed these items have developed the business processes necessary to properly stock the products, manage the inventory, properly associate prescriptions for DMEPOS items with appropriate coding under the CMS coding system, generate the regulatory paperwork for delivery of the item to the patient and generate the necessary forms for submission to the reimbursement agency such as Medicare, Medicaid, or private insurance.

Healthcare providers have a need to dispense DMEPOS items as an adjunct service to their medical practice, without having to individually develop all of the business processes which suppliers have developed previously and without the labor and overhead costs associated with those types of suppliers. Healthcare providers need to be able supply the patient with DMEPOS products that the healthcare provider has evaluated and knows to be appropriate for the patient's particular diagnosis and indications. It would be a significant advantage for the patient if the healthcare provider could provide the DMEPOS item at the time of initial diagnosis and treatment of the patient. This would allow the healthcare provider to properly fit the item to the patient and instruct the patient on the proper use of the item. For the patient, this would minimize the hassle of having to go to other locations to complete the diagnosis and treatment, and would generally result in better continuity of care.

### SUMMARY

The above and other needs are met by an integrated and automated medical product/supply dispensing, billing and inventory management system for use in the health care environment. The system includes several core functional software applications that communicate either through a networked information system architecture, or which reside on a single, general purpose computer workstation. In addition to the information system applications, the system includes a product/supply inventory management system which in general provides limited access to certain medical products or supplies and captures key information in the course of dispensing those products or supplies that subsequently enables the inventory management and billing functions of the system to operate.

Some preferred embodiments provide an inventory management system for managing information regarding medical items dispensed in conjunction with medical treatment of a patient. The medical items are stored in storage containers in a storage structure which may be in a medical facility. The inventory management system includes inventory sensors disposed on the storage structure in association with a corresponding one of the storage containers. Each inventory sensor senses removal or addition of medical items from or to the storage container associated with the inventory sensor, and generates item inventory signals indicative of the removal or addition.

The inventory management system also includes inventory indicia devices disposed on the storage structure, where each inventory indicia device is associated with a corresponding one of the storage containers in the storage structure. Each inventory indicia device provides a visual indication that a medical item is to be added to or removed from the storage container associated with the inventory indicia device. Each inventory indicia device may provide a visual indication by flashing a light or displaying a number near its associated storage container.

The inventory management system also includes a customer computer in communication with the inventory sensors and the inventory indicia devices. An inventory application, which may be running the customer computer, includes computer-executable instructions for receiving item replenishment information, for processing the item replenishment information to determine a number of medical items to be added to the storage containers to replenish inventory, and for activating the inventory indicia devices to visually indicate that medical items are to be added to the storage containers associated with the activated inventory indicia devices.

The storage structure may be a room, a cabinet, or a box. The storage containers may be bins, shelves, or drawers within the storage structure.

In some embodiments, the inventory application also receives item dispensing information, processes the item dispensing information to determine a number of medical items to be removed from the storage containers to be dispensed to a patient or used in a medical procedure, and activates the inventory indicia devices to visually indicate that medical items are to be removed from the storage containers associated with the activated inventory indicia devices.

In some embodiments, the inventory application runs on a service provider computer that is in communication with the customer computer through a communication network, and the item replenishment information is communicated over the communication network.

In some embodiments, the inventory sensors may include weight sensors attached to the storage structure, RFID sensors for sensing the presence of RFID tags attached to medical items in the inventory space, a bar code reader for scanning barcodes disposed on medical items, or a digital camera for capturing images of medical items.

Some embodiments of the inventory management system include a service provider computer and a customer computer that communicate with each other via a communication network. A treatment protocol application, which may be running on the service provider computer, accesses a listing of guidelines for a physician to follow in diagnosing and treating a medical condition of the patient, accesses a listing of medical items from which the physician may select to be dispensed for treating a diagnosed medical condition, generates item dispensing information based on a selection of a medical item from the listing of medical items, and communicates the item dispensing information via the communication network.

An inventory application, which may be running on the customer computer, receives the item dispensing information from the communication network, processes the item dispensing information to determine a number of medical items to be removed from the storage containers to be dispensed for treatment of the patient, and activates the inventory indicia devices to visually indicate that medical items are to be removed from the storage containers associated with the activated inventory indicia devices. The item dispensing information may include patient information indicating an identity of the patient for whom the medical items are to be dispensed.

In some embodiments, the inventory management system includes a physician interface computer that communicates with the service provider computer via the communication network. The physician interface computer includes a display device on which the listing of guidelines and the listing of medical items are displayed, and an input device for inputting a selection of a medical item from the listing of medical items.

In some embodiments, the treatment protocol application is executed on the service provider computer and the inventory application is executed on the customer computer.

In some embodiments, the treatment protocol application or the inventory application includes instructions for verifying that a medical item selected for dispensing is appropriate for treatment of a particular diagnosed medical condition.

### BRIEF DESCRIPTION OF THE DRAWINGS

Further advantages of the invention are apparent by reference to the detailed description in conjunction with the figures, wherein elements are not to scale so as to more clearly show the details, wherein like reference numbers indicate like elements throughout the several views, and wherein:

FIG. 1 is a functional block diagram of an embodiment of a medical product/supply dispensing, billing and inventory management system;

FIGS. 2 and 3 are flowcharts describing the operation of an embodiment of a medical product/supply dispensing, billing and inventory management system; and

FIG. 4 depicts a typical workflow for the provision of DMEPOS items based on traditional practices.

### DETAILED DESCRIPTION

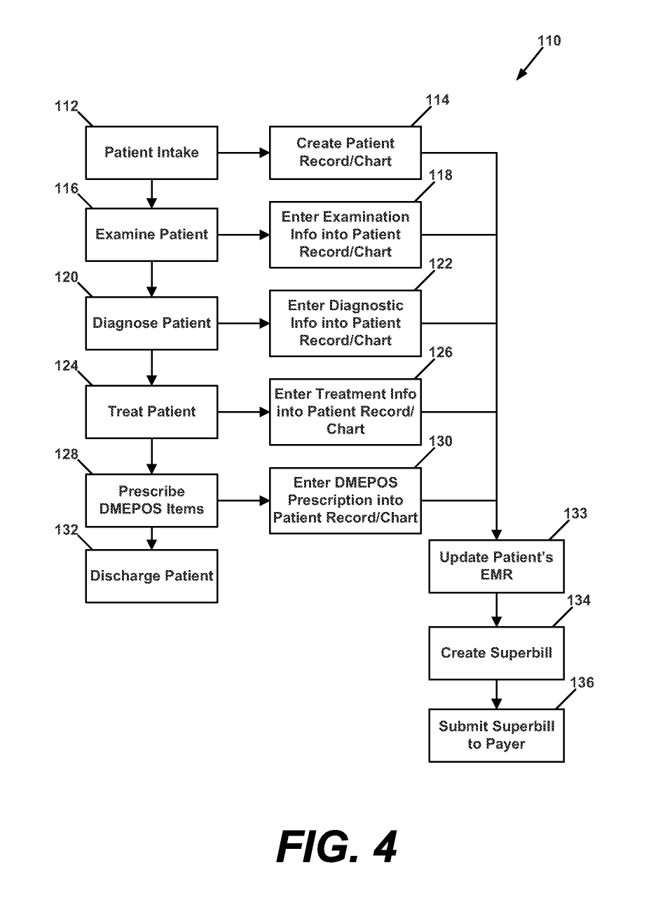

A typical workflow for the provision of DMEPOS items under traditional practices is depicted in FIG. 4. Generally, a patient follows a treatment path 110 which typically begins with the patient arriving at a treatment facility with a medical condition and ends with the patient being discharged or leaving the treatment facility. Examples of treatment facilities include a hospital emergency department, a physician's office, a clinic, and a therapy office. The first step in the treatment path 110 is typically patient intake (step 112) in which information concerning the patient is recorded and a patient record/chart (or face sheet) is created as an output (step 114). Typically, patient intake (step 112) includes collecting of basic demographic and medical information about the patient, as well as payment responsibility information, such as insurance information (either private insurance or information regarding participation in a government program such as Medicare/Medicaid). This information is recorded in the patient record/chart, which is typically created in electronic form in a pre-existing information system resident in the treatment facility.

Following patient intake (step 112) is the examination of the patient(step 116). In this step, the appropriate healthcare provider examines the patient, takes a patient history, and reviews the symptoms. The examination (step 116) may also include other diagnostic activities such as lab work and imaging that assist the provider in making an accurate diagnosis. Examination information is typically recorded into the patient record/chart (step 118). The healthcare provider then makes a diagnosis of the patient (step 120), and the diagnostic information is also entered into the patient record/chart (step 122).

After diagnosis (step 120) and entering the diagnostic information in the patient record/chart (step 122), the next step is typically treatment of the patient (step 124). In the context of this invention, treatment of the patient typically includes the healthcare provider prescribing or providing a DMEPOS item, such as an orthotic, to facilitate treatment of the diagnosed condition (step 128). Information regarding the treatment, including DMEPOS prescription information, is entered into the patient record/chart (steps 126 and 130), and this information is entered in the patient's Electronic Medical Records (step 133).

The final step in the treatment Path 110, is for the patient to be discharged or released (step 132). The information entered into the patient record/chart at each step in the treatment path 110 is used to supply information to create a medical complaint bill reflecting all of the billable services provided in the process (step 134). This information is then uploaded to the appropriate billing software for submittal to payer (step 136).

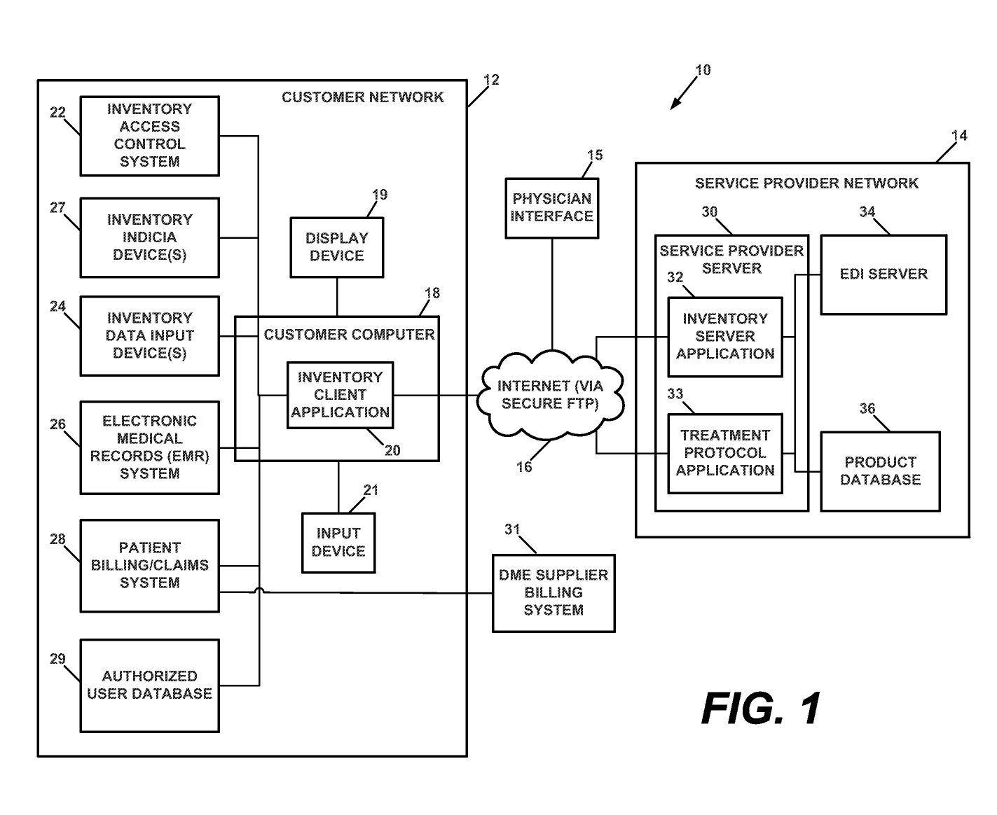

FIG. 1 depicts an embodiment of a medical product/supply dispensing, billing and inventory management system 10. As described in more detail hereinafter, the system 10 provides computer-implemented tools and processes for managing an inventory of medical products/supplies, for dispensing such products/supplies to patients, and for billing a payer for the dispensed products/supplies. However, one skilled in the art will recognize that the system 10 may be used to manage inventories of other components and materials in medical and nonmedical applications. Thus, the embodiments described herein are not limited only to medical product/supply inventory, dispensing, and billing applications.

As shown in FIG. 1, the system 10 includes a customer computer network 12 and a service provider computer network 14 which communicate with each other via a communication network 16 such as the Internet. As the term is used herein, “customer” generally refers to a medical facility where medical diagnostic and treatment procedures are performed, such as a hospital, outpatient surgical center, physician's office, clinic, or therapy office. The term “customer” may also refer to any consumer of products/supplies that are inventoried and managed using the system described herein. Accordingly, one or more of the components of the customer computer network 12 may be located within a hospital, clinic, doctor's office, or other medical facility.

The customer computer network 12 includes a customer computer 18 that is operable to communicate through the Internet 16 with the service provider computer network 14. The customer computer 18 may be, for example, a desktop computer, laptop computer, tablet computer, or smart phone. An inventory client application 20 is loaded on the customer computer 18 to provide some or all of the inventory management, dispensing, and billing functions described herein. The customer computer 18 and the client application 20 are in communication with an inventory access control system 22, one or more inventory sensors 24, an Electronic Medical Records (EMR) computer system 26, and a patient billing/claims computer system 28. A user input device 21, such as a keyboard or mouse or touchpad, is preferably provided as a component of the customer computer 18. In a preferred embodiment, the customer computer 18 also includes a display device 19 within the inventory space on which the inventory client application 20 displays information regarding inventory transactions. In some embodiments, the display device 19 and user input device 21 are combined as a touch screen device.

The inventory access control system 22 comprises a keypad, magnetic stripe reader (card swipe), proximity reader, RFID tag reader, biometric sensor device, a digital camera and facial recognition software, or other entry-access device that authorized customer personnel use to gain access to a medical product/supply inventory space. The inventory space may be a secured, limited-access location in the customer facility in which DMEPOS items are stored. In preferred embodiments, the inventory access control system 22 communicates with the customer computer 18 via a wired or wireless network connection.

As the term is used herein, an “inventory space” may be a secured, limited access inventory room or supply room. Alternatively, an inventory space may be a storage structure, such as cabinet, box, cage or other enclosure. In some embodiments, multiple inventory spaces may be provided in a single room. For example, there may be one secured cabinet containing DMEPOS items and another secured cabinet in the same supply room containing surgical implant items. In some embodiments, the inventory space may be an unsecured space into which anyone may enter or gain access.

As the term is used herein, a “storage container” may be a bin, drawer, shelf or other receptacle in which medical items are stored. Typically, there are multiple storage containers in an inventory space.

The inventory sensors 24 are devices which sense the removal or addition of DMEPOS items from or to the inventory space. In one embodiment, the inventory sensors 24 are weight sensors attached to storage bins within the inventory space. In this embodiment, the inventory sensors 24 sense that one or more items have been added to a bin based on an increase in weight of the bin, and that one or more items have been removed from the bin based on a decrease in weight of the bin. With this system, particular bins are designated to hold particular items, so that a change in weight of the bin can be associated with a change in inventory of the corresponding item.

In another embodiment, the inventory sensors 24 are RFID sensors which sense the presence of RFID tags attached to DMEPOS items within the inventory space. When an RFID tag on an item is within range of the RFID sensors, the tagged item is designated as being in inventory. Conversely, when the RFID tag on an item is outside the range of the RFID sensors, the tagged item is designated as being removed from inventory. In this embodiment, since the RFID tags identify the items to which they are attached, there is no need to designate particular bins or locations for each item within the inventory space.

In another embodiment, the inventory sensors 24 are RFID sensors which sense the presence of RFID tags attached to DMEPOS items when in range of the sensors. When an RFID tag on an item is within range of the RFID sensors, the tagged item is designated as being removed from inventory. Conversely, when the RFID tag on an item is outside the range of the RFID sensors, the tagged item is designated as being in inventory. In this embodiment, since the RFID tags identify the items to which they are attached, there is no need to designate particular bins or locations for each item within the inventory space.

In yet another embodiment, the inventory sensors 24 comprise one or more digital imaging devices, such as still cameras or video cameras, that capture images of items as the items are removed from or placed into the inventory space. In this embodiment, each item is marked with a distinctive identifier, such as a bar code, QR code, or other symbol, that uniquely identifies the item. The imaging devices are positioned such that their field of view will encompass an area through which items must pass as the items are removed from or added to the inventory space. Movement within the field of view triggers the imaging devices to capture multiple images of the items, such that at least one of the captured images may show the distinctive identifier on each item. Software executed on the customer computer 18, either as a module of the inventory client application 20 or a separate application, processes the image of the distinctive identifier (such as by “reading” the bar code or QR code) and provides identification information indicating which item is captured in the image.

In the embodiments discussed above, the inventory sensors 24 are substantially automatic. That is, they detect the addition and removal of items to and from inventory without human interaction. In a third embodiment, human interaction is required. In this embodiment, the inventory sensors 24 are barcode readers, and when a DMEPOS item is added to or removed from inventory, this is logged by scanning a barcode attached to the item.

The EMR computer system 26 comprises one or more computers that store and manage records regarding the status of patients receiving treatment in a medical facility. Generally, a patient's status is either admitted to the facility, discharged from the facility, or transferred to another department, location or facility.

In preferred embodiments, an inventory indicia device 27 is provided for each bin, shelf or drawer in the inventory space. These devices 27 preferably comprise an LED or LCD or other lighted display device disposed adjacent each bin, shelf and drawer. Depending on the current mode of operation, these devices 27 may provide a display of the number of items currently in each associated bin, shelf or drawer, or they may display a number of items that are to be added to the associated bin, shelf or drawer during a replenishment process as describe in more detail hereinafter. These indicia devices 27 are preferably controlled by the customer computer 18 based on the executed instructions of the inventory application 20.

The billing/claims computer system 28 comprises one or more computers that store and manage records regarding the billing for services rendered and DMEPOS items dispensed in examining, diagnosing, and treating patients. In preferred embodiments, this system 28 generates bills (in paper or electronic form) which are sent to patients, and generates claims which are sent to private insurers and Medicare/Medicaid. In some embodiments, the billing/claims computer system 28 is operable to communicate with a DMEPOS supplier's billing computer system 31 such as through a secure Internet connection.

With continued reference to FIG. 1, the service provider computer network 14 comprises one or more computers which store information and execute software for medical product/supply dispensing, billing and inventory management. As the term is used herein, a “service provider” may be a company that maintains inventories of DMEPOS items that are supplied to the customer to be dispensed to patients. An example of one such service provider is DeRoyal Industries, Inc. of Powell, Tennessee. Alternatively, the “service provider” may not maintain the inventory, but may provide inventory management services for another company that does maintain the inventory.

In the embodiment of FIG. 1, the service provider computer network 14 includes an inventory management server computer 30 running an inventory server application 32, an electronic data interchange (EDI) server 34, and a product inventory database 36. The EDI server 34, which may be a J.D. Edwards/Oracle server, executes programs for implementing electronic commerce transactions between the service provider network 14 and the customer network 12. The product inventory database 36 stores records indicating quantities, coding, use and application of each DMEPOS item in the service provider's inventory.

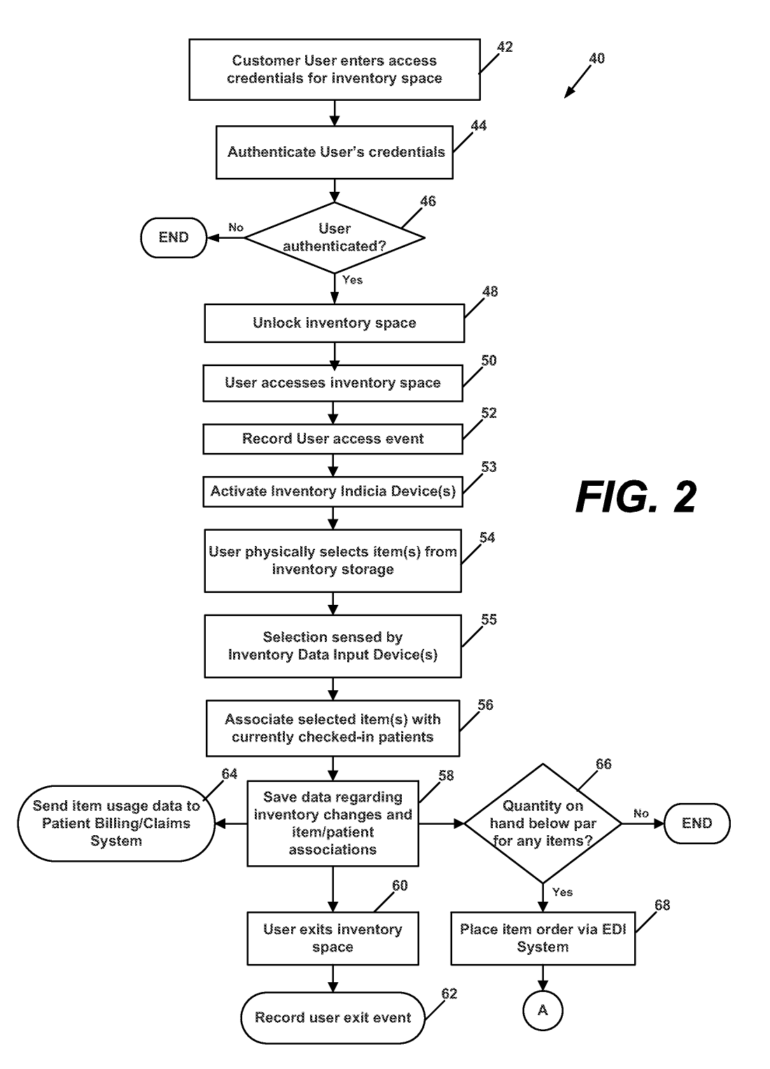

FIG. 2 depicts a process 40 for providing access to DMEPOS items in the inventory space of the customer facility, for logging removal of inventory items, and for billing the patient accordingly. Generally, the process 40 begins when a customer user enters credentials for gaining access to the inventory space (step 42). In one embodiment, this is accomplished by swiping a magnetic stripe on an ID card through a card reader of the inventory space access control system 22 (FIG. 1). In another embodiment, this is accomplished by swiping a proximity card near a proximity reader. In another embodiment, this involves passing an RFID tag near an RFID reader. In another embodiment, this involves entering a code on a keypad. In yet another embodiment, this involves a retina scan or a thumbprint scan using a biometric scanning device. In still another embodiment, this involves capturing an image of the user's face with a digital camera and processing the image using facial recognition software.

After entry of the customer user's credentials, the inventory client application 20 running on the customer computer 18 authenticates the credentials (step 44), such as by comparing the credentials to records saved in an authorized user database 29. The authorized user database 29 may be maintained on the customer network 12 or on the service provider network 14.

If the user's credentials are not authenticated (step 46), the door of the inventory space remains locked and no further action is taken, other than to log a failed entry attempt. If the user's credentials are authenticated, the inventory client application 20 executes a command to unlock a door or other access port to the inventory space (step 48) and the user may access the space (step 50). The inventory client application 20 then updates a user entry log to record this user access event, with the date and time of access and the name/ID number of the user (step 52). Additionally, the user is logged into the application 20.

In some embodiments, the inventory indicia devices 27 are activated to display to the user the quantity of each item that is to be removed from each bin, shelf or drawer in the inventory space, and the number is automatically decremented as the items are removed (step 53).

When the user selects one or more items from the shelves, bins, or drawers of the inventory space (step 54), the selection is sensed by one or more of the inventory sensors 24 and corresponding selection data is provided to the inventory client application 20 (step 55). In a preferred embodiment, a list of the selected items is then displayed on the display screen 19 for viewing by the user. In situations wherein a bill of material (BOM) has been generated for refilling a procedural tray or other kit of items and the BOM has been accessed by the inventory client application 20, the sensors 24 sense whether the correct number of items have been removed from the shelves, bins or drawers as indicated by the BOM.

Using the input device 21, the user inputs information that the inventory client application 20 uses to associate each selected item with a currently admitted or checked-in patient in the facility (step 56). To input this information, the user may select the patient's name from a list displayed on the display device 19, which list is generated from data accessed from the EMR computer system 26. Alternatively, the user may scan a barcode assigned to the patient which is attached to the patient's paperwork. In other embodiments, the patient is associated with the selected DMEPOS item by patient information included in RFID tag on the patient's chart.

The inventory client application 20 saves data regarding the removal of items from inventory (also referred to as item usage data) and data indicating the item/patient association (step 58). The item usage data and item/patient association data are sent to the patient billing/claims computer system 28 for further processing (step 64). For example, the patient billing/claims computer system 28 may use this data to submit claims for payment to appropriate insurance providers.

Based on the item usage data, the inventory client application 20 determines whether the remaining quantity of inventory items is below a predetermined minimum threshold (step 66). If so, the inventory client application 20 places an order for some quantity of the items via the Internet connection to the service provider's EDI server 34 (step 68). Alternatively, the inventory client application 20 sends an email or other electronic message to the person responsible for maintaining the inventory to remind the person to place an order.

After the user has associated the items taken from inventory with one or more patients (step 56), the user may exit the inventory space (step 60) and the inventory client application 20 logs a user exit event (step 62). In some embodiments wherein the inventory space is a room or cage, the inventory room door may not be opened to allow the user to exit until the user has done whatever is needed to complete item/patient association (step 56). In other embodiments, the door may be opened, but if the item/patient association has not been completed beforehand, an alarm sounds to remind the user to complete that task before leaving. In some embodiments, if the user leaves the inventory space without completing the item/patient association, the user will receive an email or text message reminding the user to complete the task.

By logging user access and exit events, the system 10 keeps track of all users that access the inventory space, and it associates the items removed from or added to inventory with the dates/times that each user accessed the inventory space. This provides for user accountability in the inventory process.

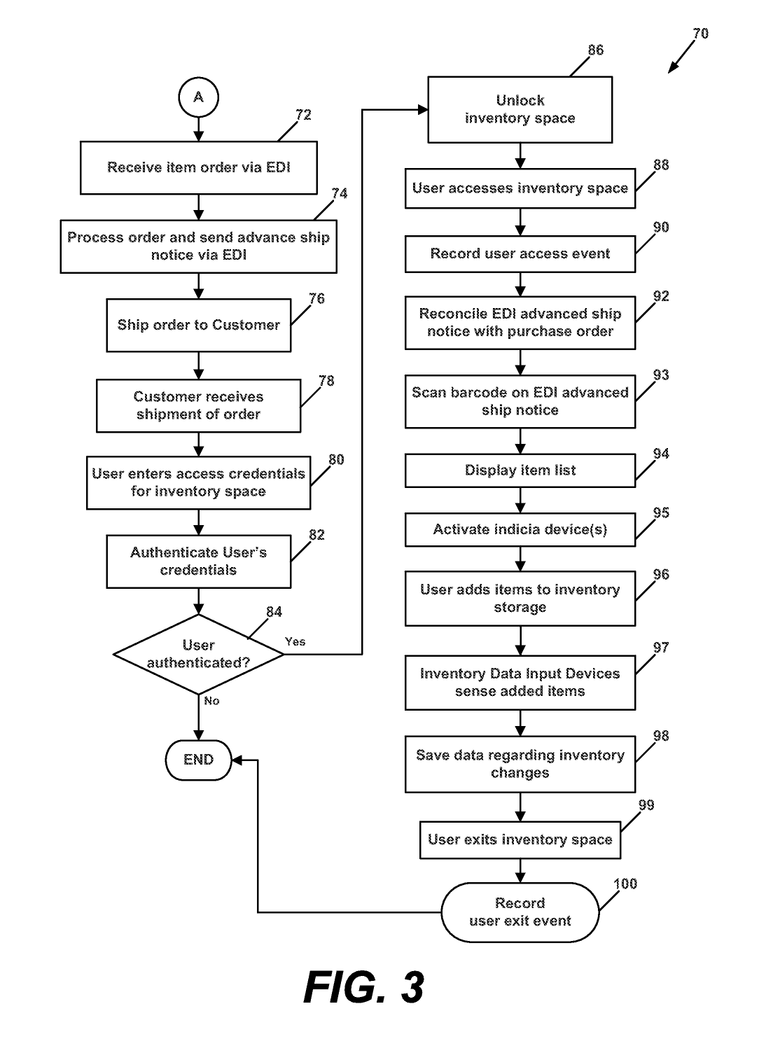

FIG. 3 depicts a preferred embodiment of a process 70 for replenishing items in the inventory space of the customer facility. Initially, the service provider's inventory server application 32 receives the order for items (step 72) that was placed by the inventory client application 20 in step 68 of FIG. 2. The inventory server application 32 passes the order to the EDI server 34. The EDI server 34 processes the order and sends an advance ship notice to the customer via the inventory server application 32 (step 74). The service provider then ships the ordered items (step 76) and the customer receives the ordered items (step 78). The customer user then takes the received items to the inventory space to restock the inventory. The customer user enters credentials for gaining access to the inventory space (step 80), such as by swiping a magnetic stripe on an ID card through a card reader of the inventory space access control system 22. The inventory client application 20 running on the customer computer 18 authenticates the user's credentials (step 82) as described above.

If the user's credentials are not authenticated (step 84), the door or entry port of the inventory space remains locked and no further action is taken, other than to log a failed entry attempt. If the user's credentials are authenticated, the inventory client application 20 executes a command to unlock the inventory space door or entry port (step 86) and the user may access the inventory space (step 88). The inventory client application 20 then updates a user entry log to record this user access event, with the date and time of access and the name/ID number of the user (step 90). Alternatively or in addition, the inventory server application 32 may update a user access log maintained on the service provider server 30.

The user reconciles the EDI advance ship notice with the purchase order that was issued in step 68 of FIG. 2 (step 92). In a preferred embodiment, this process includes scanning a barcode or other unique identifier on the EDI advance ship notice (step 93), at which point the inventory client application 20 accesses item replenishment information, such as a list of items associated with the identified EDI advance ship notice, and displays this information on the display device 19 (step 94). Based on the item replenishment information, the application 20 also activates the appropriate inventory indicia devices 27 associated with each bin, shelf, or drawer in the inventory space to indicate which bins, shelves or drawers are to receive replenishment items, and how many items are to be put in each bin, shelf or drawer (step 95). This activation may comprise turning on or lighting up indicia devices 27 that were previously in an off condition, or causing the indicia devices 27 to flash in some manner to attract the user's attention.

When the user physically adds the replenishment items to the appropriate bins, shelves, or drawers in the inventory space (step 96), the addition is automatically sensed by one or more of the inventory sensors 24 (such as by sensing additional weight in a bin or by sensing the presence of previously unlogged RFID tags on the added items) and the corresponding replenishment data is provided to the inventory client application 20 (step 97). The inventory client application 20 records the data indicating an addition of items to inventory (step 98). In a preferred embodiment, a list of the added items is displayed on the display screen 19 for viewing, confirmation, and editing by the user. When the user exits the inventory space (step 99), the inventory client application 20 logs a user exit event (step 100).

It should be appreciated that many of the steps of FIGS. 2 and 3 may be performed by the inventory server application 32, by the inventory client application 20, or by both working together. Thus, the invention is not limited to performance of the process steps by any particular application or on any particular computer system.

As shown in FIG. 1, some embodiments of the invention include a physician interface computer 15, which may be a personal computer, laptop computer, tablet/pad computer, smart phone, or other such computing device. During an examination of a patient, a physician may use the physician interface computer 15 to access and follow diagnostic/treatment protocol guidelines, record diagnostic information, record patent information, make notes, and order medical items to be dispensed to the patient as part of the patient's treatment. Preferably, the physician interface computer 15 is operable to communicate with the customer network 12 and the service provider network 14 via the Internet 16 or other communication network. In a preferred embodiment, a physician may use the physician interface computer 15 to prescribe items in inventory that a patient needs, and send the order data for the prescribed items to the inventory client application 20 running on the customer computer 18.

As alluded to above, the physician interface computer 15 may be used to access diagnostic/treatment protocol guidelines for various types of injuries, such as orthopedic injuries. Preferably, these guidelines are provided in a web-based application that may be accessed using the physician interface computer 15 via the Internet 16. In one preferred embodiment depicted in FIG. 1, the guidelines are provided by a treatment protocol application 33 running on the service provider server 30. The guidelines may include checklists and tips for diagnosis, treatment, documentation, and billing for various types of injuries. Preferably, the guidelines include a list of related medical items, such as DMEPOS items, that can be dispensed for treatment of a diagnosed injury. These items can be ordered via the service provider network 14 and the EDI server 34.

In some embodiments, the treatment protocol application 33 displays listings on the physician interface computer 15 of reimbursement billing codes and diagnostic codes associated with the types of injuries covered by the guidelines. The billing codes indicate Medicare-reimbursable amounts for the examination and for the medical items prescribed for treatment. The diagnostic codes (also referred to as ICD codes) identify the type of injury.

In a preferred embodiment, the treatment protocol application 33 also displays listings of medical items that may be ordered from the service provider for treatment of the particular diagnosed injury, with links for directly ordering the items. For example, for an orthopedic ankle injury, the treatment protocol application 33 may display a listing of available ankle braces, ankle stirrups and walking boots. The items are preferably listed in groups according a product code, such as Healthcare Common Procedure Coding System (HCPCS) codes.

In addition to ordering medical items from the service provider for replenishing inventory, preferred embodiments of the treatment protocol application 33 allow the physician to use the physician interface computer 15 to dispense an item from inventory at the doctor's office or clinic. In this embodiment, the treatment protocol application 33 generates and sends item dispensing information, which may include a product code for the item to be dispensed, a patient identifier, and a billing code to the inventory client application 20. Based on the item dispensing information, the inventory client application 20 displays information on the display device 19 indicating the item to be dispensed, the bin, shelf or drawer where the item may be found in the inventory space, and patient identification information. Preferably, the inventory client application 20 activates the appropriate inventory indicia device 27 to indicate the bin, shelf or drawer where the item is located in the inventory space.

In one embodiment, when the physician uses the treatment protocol application 33 to dispense a particular medical item for a patient, the treatment protocol application 33 verifies that the HCPCS code identifies an item that is appropriate for treatment of the injury or condition indicated by the diagnostic code entered in the patient's record. If the item is not appropriate, the treatment protocol application 33 displays a message prompting the physician to double check that the correct item is being dispensed for treatment of the particular injury.

Although some embodiments of the invention are directed to dispensing, billing and inventory management for DMEPOS items, the apparatus and methods described herein are also applicable to dispensing, billing and inventory management of other types of medical items, such as surgical implant items and items used in a catheter lab. Thus, “medical item” as that term is used herein is not limited to DMEPOS items or any other particular type of medical item.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An inventory management system for managing information regarding medical items dispensed in conjunction with medical treatment of a patient, wherein the medical items are disposed in a plurality of storage containers in a storage structure, the inventory management system comprising:
a plurality of inventory sensors disposed on the storage structure, each inventory sensor associated with a corresponding one of the storage containers, wherein each inventory sensor is operable to:
sense removal of one or more medical items from the storage container associated with the inventory sensor;
sense addition of one or more medical items to the storage container associated with the inventory sensor; and
generate item inventory signals indicative of the removal or addition of medical items from or to the storage container associated with the inventory sensor;
a plurality of inventory indicia devices disposed on the storage structure, each inventory indicia device associated with a corresponding one of the storage containers in the storage structure, wherein each inventory indicia device is operable to provide a visual indication that a medical item is to be added to or removed from the storage container associated with the inventory indicia device;
a customer computer in communication with the inventory sensors and the inventory indicia devices; and
one or more inventory applications in communication with the customer computer, the one or more inventory applications including computer-executable instructions which, when executed:
receive item replenishment information;
process the item replenishment information to determine a number of medical items to be added to one or more of the storage containers to replenish inventory; and
activate one or more of the inventory indicia devices to visually indicate that a medical item is to be added to the one or more storage containers associated with the one or more inventory indicia devices activated.

2. The system of claim 1 wherein the one or more inventory applications include computer-executable instructions which, when executed:
receive item dispensing information;
process the item dispensing information to determine a number of medical items to be removed from one or more of the storage containers to be dispensed to a patient or used in a medical procedure; and
activate one or more of the inventory indicia devices to visually indicate that one or more medical items are to be removed from the one or more storage containers associated with the one or more inventory indicia devices activated.

3. The system of claim 1 wherein one or more of the inventory applications are executed on the customer computer.

4. The system of claim 1 wherein one or more of the inventory applications are executed on a service provider computer that is in communication with the customer computer through a communication network, wherein the item replenishment information is communicated over the communication network.

5. The system of claim 1 wherein the plurality of inventory sensors include one or more of:
a weight sensor attached to the storage structure;
an RFID sensor for sensing the presence of an RFID tag attached to a medical item in the inventory space;
a bar code reader for scanning a barcode on a medical item; and
a digital camera for capturing an image of a medical item.

6. The system of claim 1 further comprising:
a display device connected to the customer computer and viewable by the user; and
the one or more inventory applications including computer-executable instructions which, when executed, cause the item replenishment information to be displayed on the display device for viewing by the user.

7. The system of claim 1 further comprising:
a service provider computer associated with a service provider that provides medical items for replenishing the inventory of medical items in the storage structure, the service provider computer in communication with the customer computer via a communication network; and
an electronic data interchange server in communication with the service provider computer, the electronic data interchange server for processing orders for medical items placed with the service provider.

8. The system of claim 7 wherein the one or more inventory applications include:
computer-executable instructions which, when executed, update records indicating a count of medical items in one or more of the storage containers in the storage structure;
computer-executable instructions which, when executed, determine that the count of medical items in one or more of the storage containers has dropped below a predetermined threshold; and
computer-executable instructions which, when executed, automatically place an order communicated through the communication network to the electronic data interchange server for medical items to replenish the inventory in the storage structure when the count has dropped below the predetermined threshold.

9. The system of claim 1 wherein the storage structure comprises a room, a cabinet, or a box.

10. The system of claim 1 wherein the storage containers comprise bins, shelves, or drawers within the storage structure.

11. The system of claim 1 wherein each inventory indicia device provides a visual indication comprising one or more of a flashing light and a numerical display.

12. The system of claim 1 further comprising:
an inventory access control system in communication with the customer computer, the inventory access control system for controlling access to the storage structure, the inventory access control system further for receiving credential information from a user seeking access to the storage structure; and
the one or more inventory applications include computer-executable instructions which, when executed, process the credential information to determine whether the user is authorized to access the storage structure, and communicate with the inventory access control system to permit user access to the storage structure when the credential information indicates that the user is so authorized.

13. An inventory management system for managing information regarding medical items to be dispensed in conjunction with medical treatment of a patient, wherein the medical items are disposed in a plurality of storage containers in a storage structure, the inventory management system comprising:
a service provider computer operable to communicate via a communication network;
a treatment protocol application in communication with the service provider computer, the treatment protocol application including computer-executable instructions which, when executed:
access a listing of guidelines for a physician to follow in diagnosing and treating a medical condition of the patient;
access a listing of medical items from which the physician may select to be dispensed for treating a diagnosed medical condition;
generate item dispensing information based on a selection of a medical item from the listing of medical items; and
communicate the item dispensing information via the communication network;
a customer computer in communication with the service provider computer via the communication network;
a plurality of inventory indicia devices disposed on the storage structure and in communication with the customer computer, each inventory indicia device associated with a corresponding one of the storage containers, wherein each inventory indicia device is operable to provide a visual indication that one or more medical items are to be removed from the storage container associated with the inventory indicia device; and
one or more inventory applications in communication with the customer computer, the one or more inventory applications including computer-executable instructions which, when executed:
receive the item dispensing information from the communication network;
process the item dispensing information to determine a number of medical items to be removed from one or more of the storage containers to be dispensed for treatment of the patient; and
activate one or more of the inventory indicia devices to visually indicate that one or more medical items are to be removed from the one or more storage containers associated with the one or more inventory indicia devices activated.

14. The system of claim 13 further comprising a physician interface computer in communication with the service provider computer via the communication network, the physician interface computer including a display device on which the listing of guidelines and the listing of medical items are displayed, and an input device for inputting the selection of a medical item from the listing of medical items.

15. The system of claim 13 wherein the item dispensing information includes patient information indicating an identity of the patient for whom the one or more medical items are to be dispensed.

16. The system of claim 13 wherein the treatment protocol application is executed on the service provider computer.

17. The system of claim 13 wherein at least one of the one or more inventory applications is executed on the customer computer.

18. The system of claim 13 wherein one or more of the treatment protocol application and the inventory application include instructions for verifying that a medical item selected for dispensing is appropriate for treatment of a diagnosed medical condition.

19. The system of claim 13 further comprising:
an inventory access control system in communication with the customer computer, the inventory access control system for controlling access to the storage structure, the inventory access control system further for receiving credential information from a user seeking access to the storage structure; and
the one or more inventory applications include computer-executable instructions which, when executed, process the credential information to determine whether the user is authorized to access the storage structure, and communicate with the inventory access control system to permit user access to the storage structure when the credential information indicates that the user is so authorized.
